(19) United States
(12) Patent Application Publication
(10) Pub. No.: US 2025/0323500 A1
(43) Pub. Date: Oct. 16, 2025

(54) METHOD FOR COLLABORATIVE SOURCE APPORTIONMENT OF VOCS, PRODUCT, MEDIUM, AND DEVICE

(71) Applicant: Jinan University, Guangzhou City (CN)

(72) Inventors: Mei LI, Guangzhou City (CN); Yongjiang XU, Guangzhou City (CN); Bin YUAN, Guangzhou City (CN); Min SHAO, Guangzhou City (CN)

(21) Appl. No.: 19/169,235

(22) Filed: Apr. 3, 2025

(30) Foreign Application Priority Data
Apr. 11, 2024 (CN) 202410430599.6

(51) Int. Cl. H02J 3/00; G01N 33/00; H01J 49/00

(52) U.S. Cl. CPC H02J 3/00; G01N 33/0047; H01J 49/0036

(57) ABSTRACT

A method for collaborative source apportionment of volatile organic compounds (VOCs), a product, a medium, and a device are provided. The method includes: establishing a single particle classification model; training and optimizing the single particle classification model with a local pollution library, and analyzing pollution sources of single particle mass spectrometric data to be apportioned to obtain a time series of the pollution sources contributing to the particulate matter (PM); obtaining VOCs factors of the pollution sources and a time series thereof; performing correlation calculation on the time series of the pollution sources contributing to the PM and the time series of the VOCs factors to obtain a correlation coefficient; and attributing the PM and the VOCs factor having the correlation coefficient higher than a set threshold to a same pollution source, and identifying a common pollution source of the PM and the VOCs.

### CROSS REFERENCE TO RELATED APPLICATION

This patent application claims the benefit and priority of Chinese Patent Application No. 202410430599.6, filed with the China National Intellectual Property Administration on Apr. 11, 2024, the disclosure of which is incorporated by reference herein in its entirety as part of the present patent application.

### TECHNICAL FIELD

The present disclosure relates to the technical field of source apportionment, and in particular, to a method for collaborative source apportionment of VOCs, a product, a medium, and a device.

### BACKGROUND

In the atmospheric environment, a pollution source may emit particulate matter (PM) and volatile organic compounds (VOCs) simultaneously. In past atmospheric pollution source apportionment, generally, source apportionment is performed on the PM and the VOCs separately rather than from a unified perspective.

Collaborative control of PM and ozone is required in the atmospheric pollution prevention and control process. VOCs are key precursors of the ozone. Therefore, finding a common contributing source of PM and VOCs is of great significance for focusing and controlling an important source. However, there is no method for identifying a common pollution source of PM and VOCs at present. The source apportionment can only be performed on PM and VOCs separately. Nowadays, we only perform the source apportionment of VOCs and PM using different models. There is no algorithm model capable of simultaneously apportioning the PM and the VOCs. Therefore, the collaborative source apportionment of the PM and the VOCs cannot be realized, and it is impossible to identify the common pollution source of the PM and the VOCs to provide technical support for the collaborative control of pollution sources in the atmospheric environment.

### SUMMARY

An objective of the present disclosure is to provide a method for collaborative source apportionment of VOCs, a product, a medium, and a device that can realize collaborative source apportionment of PM and VOCs and identify a common pollution source of the PM and the VOCs to provide technical support for the collaborative control of pollution sources in the atmospheric environment.

To achieve the above objective, the present disclosure provides the following solutions.

In an aspect, the present disclosure provides a method for collaborative source apportionment of VOCs, including:
monitoring, by a single particle aerosol mass spectrometry, a particulate matter (PM) in an atmospheric environment at a target area for a period of time, to obtain target single particle mass spectrometric data;
monitoring, by an on-line VOCs monitoring instrument, VOCs in the atmospheric environment in the target area for the period of time, to obtain target VOCs monitoring data;
inputting the target single particle mass spectrometric data and the target VOCs monitoring data into a pollution source identifying device to identify a common pollution source of the PM and the VOCs in the target area for the period of time, wherein the source identifying device comprises a processor and a memory having an optimized single particle classification model and a positive matrix factorization (PMF) model stored therein and is configured for;
inputting the target single particle mass spectrometric data to the optimized single particle classification model for apportionment, and analyzing pollution sources of the target single particle mass spectrometric data using the optimized single particle classification model to output a time series of the pollution sources contributing to the PM; wherein the optimized single particle classification model is a deep learning model based on a one-dimensional convolutional neural network, a self-attention mechanism, and a multi-layer perceptron;
inputting the target VOCs monitoring data to the positive matrix factorization (PMF) model for apportionment, and outputting VOCs factors of the pollution sources and a time series of the VOCs factors by the PMF model;
performing correlation calculation on the time series of the pollution sources contributing to the PM and the time series of the VOCs factors to obtain a correlation coefficient; and
attributing the PM and the VOCs factor having a correlation coefficient higher than a predetermined threshold to a same pollution source, thereby identifying the common pollution source of the PM and the VOCs in the target area for the period of time.

Optionally, the optimized single particle classification model is obtained by following step:
obtaining a local pollution library, wherein the local pollution library comprises single particle mass spectrometric data of a known pollution source;
establishing a single particle classification model, wherein the single particle classification model is a deep learning model based on a one-dimensional convolutional neural network, a self-attention mechanism, and a multi-layer perceptron;
training and optimizing the single particle classification model with the local pollution library to obtain the optimized single particle classification model.

Optionally, the method further comprises the step of adjusting an electrical power supplied by an industrial power grid to a factory corresponding to a category of the common pollution source to limit the generation of the PM and the VOCs.

Optionally, the one-dimensional convolutional neural network, the self-attention mechanism, and the multi-layer perceptron are connected in sequence;
the one-dimensional convolutional neural network is configured to extract a local feature from the input single particle mass spectrometric data;
the self-attention mechanism is configured to calculate a plurality of features extracted by the one-dimensional convolutional neural network; and
the multi-layer perceptron is configured to receive series data processed by the self-attention mechanism, put the series data through an input layer, a hidden layer, and an output layer, and output a final PM classification result.

Optionally, calculating, by the self-attention mechanism, a plurality of features extracted by the one-dimensional convolutional neural network specifically includes:
for the plurality of features extracted by the one-dimensional convolutional neural network, calculating Query, Key, and Value values by linear variation;
calculating a similarity between the Query and Key values based on a dot product formula;
normalizing the similarity between the Query and Key values by a Softmax function to obtain a weight of attention; and
performing weighted summation on the Value values with the weight of the attention to obtain a final output, wherein the final output is one-dimensional series information.

Optionally, when training and optimizing the single particle classification model with the local pollution library, a categorical cross-entropy suitable for a multi-classification task is used as a loss function.

Optionally, the performing correlation calculation on the time series of the pollution sources contributing to the PM and the time series of the VOCs factors to obtain a correlation coefficient specifically includes:
performing correlation calculation on the time series of the pollution sources contributing to the PM and the time series of the VOCs factors by a Pearson correlation coefficient calculation formula to obtain a Pearson correlation coefficient.

In another aspect, the present disclosure provides a computer program product, including a computer program which, when executed by a processor, implements steps of the method for collaborative source apportionment of VOCs.

In another aspect, the present disclosure further provides a computer-readable storage medium, storing a computer program which, when executed by a processor, implements steps of the method for collaborative source apportionment of VOCs.

In yet another aspect, the present disclosure provides a computer device, including a memory, a processor, and a computer program stored on the memory and runnable on the processor, where the processor is configured to execute the computer program to perform the steps of the method for collaborative source apportionment of VOCs.

According to specific embodiments provided in the present disclosure, the present disclosure has the following technical effects:

In the present disclosure, the deep learning model based on the one-dimensional convolutional neural network, the self-attention mechanism, and the multi-layer perceptron is utilized to analyze the sources of the PM in the atmospheric environment, thereby obtain the time series of the pollution sources contributing to the PM, and then the time series of the VOCs factors of the on-line monitored VOCs is obtained by the PMF model, and correlation calculation is performed on the time series. When the correlation coefficient is higher than a set value, it indicates that two groups of source apportionment results have a correlation in time series and can be attributed to the same pollution source. Thus, the PM and the VOCs having the correlation coefficient higher than the set value are attributed to the same pollution source, thereby realizing the collaborative source apportionment, identifying the common pollution source of the PM and the VOCs. It provides technical support for the collaborative control of pollution sources in the atmospheric environment.

### BRIEF DESCRIPTION OF THE DRAWINGS

To describe the technical solutions in the embodiments of the present disclosure or in the prior art more clearly, the accompanying drawings required for the embodiments are briefly described below. Apparently, the accompanying drawings in the following description show merely some embodiments of the present disclosure. Others may still derive more accompanying drawings from following accompanying drawings without creative efforts.

FIG. 1 is a flowchart of a method for collaborative source apportionment of VOCs provided by Example 1 of the present disclosure;

FIG. 2 is a flowchart of running of the present disclosure.

### DETAILED DESCRIPTION OF THE EMBODIMENTS

The technical solutions of the embodiments of the present disclosure are clearly and completely described below with reference to the accompanying drawings. Apparently, the described embodiments are merely a part rather than all of the embodiments of the present disclosure. All other embodiments obtained by a person of ordinary skill on the basis of the described embodiments without creative efforts belong to this invention.

An objective of the present disclosure is to provide a method for collaborative source apportionment of VOCs, a product, a medium, and a device that can realize collaborative source apportionment of PM and VOCs and identify a common pollution source of the PM and the VOCs to provide technical support for the collaborative control of pollution sources in the atmospheric environment.

To make the above objective, features, and advantages of the present disclosure clearer and more comprehensible, the present disclosure will be further described in detail below with reference to the accompanying drawings and the specific examples.

### EXAMPLE 1

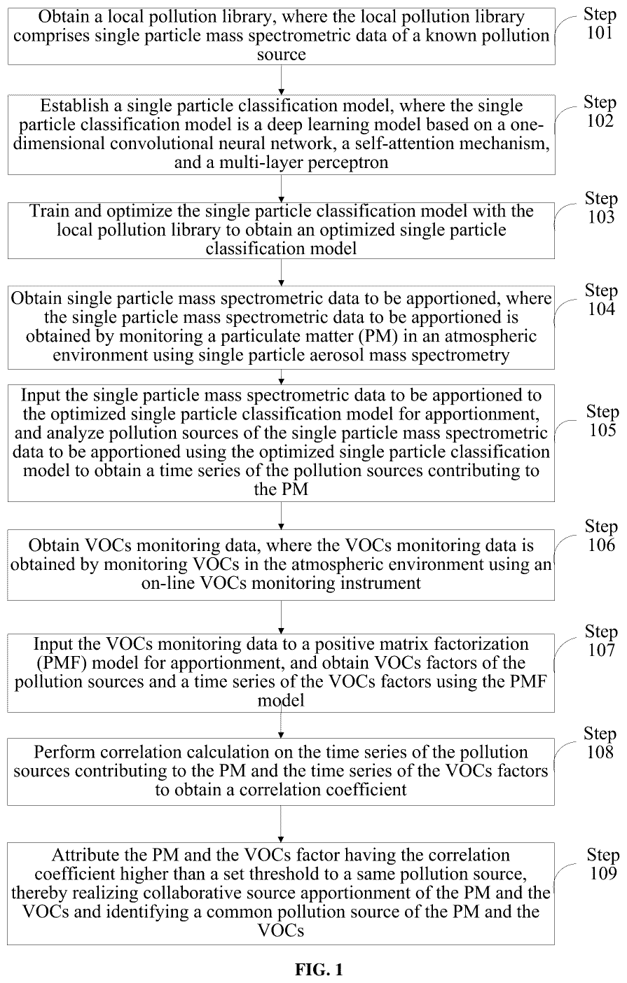

As shown in FIG. 1, Example 1 provides a method for collaborative source apportionment of VOCs, including the following steps.

In step 101, a local pollution library is obtained, where local pollution library includes single particle mass spectrometric data of a known pollution source.

In step 102, a single particle classification model is established, where the single particle classification model is a deep learning model based on a one-dimensional convolutional neural network, a self-attention mechanism, and a multi-layer perceptron.

In step 102, the one-dimensional convolutional neural network, the self-attention mechanism, and the multi-layer perceptron are connected in sequence. The one-dimensional convolutional neural network is configured to extract a local feature from the input single particle mass spectrometric data. The self-attention mechanism is configured to calculate features extracted by the one-dimensional convolutional neural network. The multi-layer perceptron is configured to receive series data processed by the self-attention mechanism, put the series data through an input layer, a hidden layer, and an output layer, and output a final PM classification result.

Calculating features extracted by the one-dimensional convolutional neural network specifically and the self-attention mechanism includes the following steps.

For features extracted by the one-dimensional convolutional neural network, Query, Key, and Value values are calculated by linear variation.

A similarity between the Query and Key values is calculated based on a dot product formula.

The similarity between the Query and Key values is normalized by a Softmax function to obtain a weight of attention.

Weighted summation is performed on the Value values with the weight of the attention to obtain a final output, which is one-dimensional series information.

In step 103, the single particle classification model is trained and optimized with the local pollution library to obtain an optimized single particle classification model.

In step 103, when training and optimizing the single particle classification model with the local pollution library, a categorical cross-entropy suitable for a multi-classification task is used as a loss function.

In step 104, single particle mass spectrometric data to be apportioned which is obtained by monitoring a PM in an atmospheric environment using single particle aerosol mass spectrometry.

In step 105, the single particle mass spectrometric data to be apportioned is input to the single particle classification model for apportionment, and pollution sources are analyzed. Then, obtain a time series of the pollution sources contributing to the PM.

In step 106, the VOCs monitoring data is obtained by monitoring VOCs in the atmospheric environment using an on-line VOCs monitoring instrument.

In step 107, the VOCs monitoring data is input to a PMF model for apportionment, and VOCs factors of the pollution sources and a time series of the VOCs factors are obtained using the PMF model.

In step 108, correlation calculation is performed on the time series of the pollution sources contributing to the PM and the VOCs factors.

Step 108 specifically includes the following step:

Correlation calculation is performed on the time series of the pollution sources contributing to the PM and the time series of the VOCs factors by a Pearson correlation coefficient calculation formula to obtain a Pearson correlation coefficient.

In step 109, the PM and the VOCs factor having a correlation coefficient higher than a set threshold are attributed to a same pollution source, thereby realizing collaborative source apportionment of the PM and the VOCs. Then, identify a common pollution source of the PM and the VOCs.

As described above, the pollution sources of each single particle mass spectrometric data from local pollution library in step 101 are known. The known sources of pollution can specifically be factories, or even different processes or discharge outlets within factories. Therefore, these known pollution sources can be classified, based on the industrial types to which the pollution source belongs or industry classification, into different pollution source categories. For example, the industrial types may be broad types such as textile industry, mining industry, chemical production industry, transportation industry, etc. Further, the industrial types may be specific types, taking mining as an example, such as coal mining, oil and gas mining, black metal mining, non-ferrous metal mining, non-metallic mining and the like (which all belongs to mining industry). The single particle classification model is trained based on single particle mass spectrometry data labeled with pollution source categories to obtain an optimized single particle classification model. The optimized single particle classification model can identify a corresponding category of pollution source, for each particle collected over a period of time.

As for VOCs, the PMF model is used to analyze the VOCs monitoring data (including concentration and time data of various VOCs species), so as to obtain several broad classes. However, existing technology requires to manually analyze the corresponding categories according to the concentration and temporal variation characteristics of VOCs species within each broad class.

The methods provided above utilize correlation analysis to establish the relationship between particulate matter and VOCs. The data (i.e. the time series of VOCs concentration) in the broad classes analyzed by PMF can be correlated with the analysis result (i.e. the time series of the pollution sources contributing to the PM) of single particle classification model. If the correlation coefficient higher than a predetermined threshold, the VOCs broad class can be considered as the same as the source category of PM, thereby determining the category of common pollution source.

The following describes the technical solutions of the present disclosure by using a specific example.

In consideration of the following aspects, the present disclosure provides a method for collaborative source apportionment of VOCs.

Although the mechanical interaction between a particle phase and a gaseous phase is unknown, based on the correlation of pollutants emitted from the same source in time series, a unified source apportionment perspective of the PM and the VOCs can be established to a certain extent, thereby effectively improving the collaborative control capability of the atmospheric pollution and realizing the common control of the PM and the VOCs. Moreover, in previous VOCs source apportionment, the PMF model developed by the U.S. Environmental Protection Agency is often used independently. In the use process of the PMF model, species need to be selected artificially, and the attribution of factors is determined. A strong subjective factor is introduced, and factors having strong homology cannot be further fine distinguished. Thus, the PMF model has limitations in the current context of increasingly refined atmospheric pollution management. The single particle aerosol mass spectrometry (SPAMS) can realize real-time and on-line monitoring on single particle aerosol directly and rapidly. Not only is the difficulty of sample pretreatment in a traditional off-line analysis method avoided, but also a single particle size, chemical components, and information thereof changing over time and spatial distribution can be obtained. By a single particle mass spectrometric source apportionment technique, the time change of the aerosol of a certain source can be obtained and then subjected to time correlation analysis with pollution source factors of the VOCs obtained in the PMF model so that a common pollution source can be identified. Moreover, the single particle mass spectrometric source apportionment technique is a method based on deep learning, which has powerful feature extraction and nonlinear identification capability and can effectively carry out refined apportionment of the pollution sources.

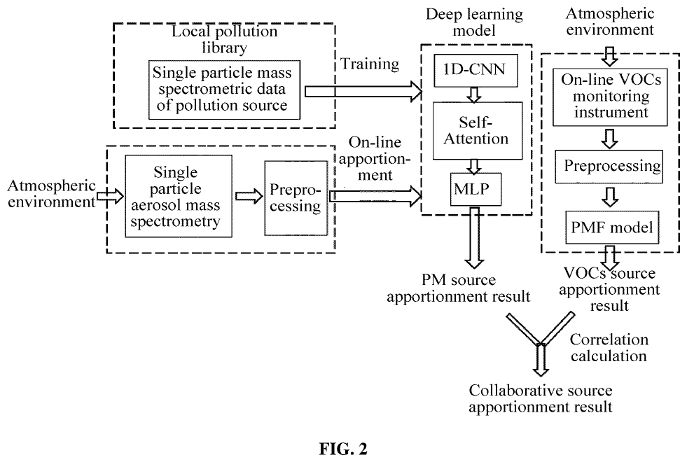

The method for collaborative source apportionment of VOCs proposed in the present disclosure based on single particle mass spectrometry. The running process of the present disclosure is as shown in FIG. 2. The present disclosure is based on a single particle aerosol source apportionment technique of a deep learning algorithm. The single particle classification model is trained with a local source library (the local pollution library). Subsequently, the sources of the PM in the atmospheric environment are analyzed to obtain a time series of pollution source contributions (the time series of the pollution sources contributing to the PM), and then the time series of the VOCs factors (a contribution spectrum) of the on-line monitored VOCs is obtained by the PMF model, and correlation calculation is performed on the time series. The PM and the VOCs factor having a high correlation coefficient are attributed to the same pollution source.

The existing PM source apportionment techniques mostly use the PMF model, and then the time series of the pollution source contributions are obtained by artificial judging of source attribution. The present disclosure uses the deep learning model based on the one-dimensional convolutional neural network, the self-attention mechanism, and the multi-layer perceptron to analyze the sources of the PM in the atmospheric environment and obtain the time series of the pollution source contributions. Theoretically, Self-Attention (the self-attention mechanism) can better identify a relationship between mass spectrometric peaks. A traditional time series neural network (long short term memory, LSTM) is used in the prior work. When there are many peaks (i.e., there is a large quantity of feature information needing to be identified), a forgetting phenomenon may occur. The forgetting phenomenon may not occur for the Self-Attention structure, and the accuracy rate of identifying the relationship of many mass spectrometric peaks can be increased. In the natural language field, the traditional time series neural network such as LSTM or CNN is used in past machine translation, but now frameworks of the Self-Attention type are mostly adopted, avoiding mistakes generated due to too long sentences (google translation). In AI applications such as ChatGPT, similar Self-Attention frameworks are also used.

The source apportionment of the PM is performed in the deep learning model which is based on the one-dimensional convolutional neural network (1D-CNN), the self-attention mechanism, and the multi-layer perceptron (MLP). The 1D-CNN, the Self-Attention, and the MLP in the present disclosure will be specifically described below.

1. 1D-CNN: for the mass spectrometric information of the atmospheric PM, the data of positive and negative spectrograms is subjected to L2 norm normalization and thus converted into a one-dimensional array with values of 0 to 1, and then the 1D-CNN is used to extract local features.

$$Y = {σ}_{r} ( W \cdot X + b ) \qquad ( 1 )$$

In formula (1), Y represents a feature extracted by the 1D-CNN, which is series data of one dimension; σᵣ represents an activation function Relu; W represents a weight matrix; X represents an input one-dimensional mass spectrometric array; and b represents an offset vector. An expression of the Relu activation function is max(0,x), which is a nonlinear function.

2. Self-Attention: for features extracted by the 1D-CNN, they are input to a self-attention module for calculation, which is mainly divided into the following steps.

### (1) Calculation of Query, Key, and Value

The series data [x₁, x₂, x₃, . . . , xₙ] from the 1D-CNN is obtained, and Query (Q), Key (K), and Value (V) are obtained by the following linear variation.

$${Q}_{i} = {x}_{i} {W}_{Q} \qquad ( 2 )$$

$${K}_{i} = {x}_{i} {W}_{K} \qquad ( 3 )$$

$${V}_{i} = {x}_{i} {W}_{V} \qquad ( 4 )$$

where W_{Q}, W_{K}, and W_{V} are weight matrices obtained by the model through learning; xᵢ represent ith data in an input series; Qᵢ represents an ith element of Query obtained by linear variation; Kᵢ represents an ith element of key obtained by linear variation; Vᵢ represents an ith element of Value obtained by linear variation.

### (2) Calculation of Similarity

This process is mainly to calculate a similarity between Q and K values based on the dot product formula.

$$Attention ( {Q}_{i} , {K}_{j} ) = \frac{{Q}_{i} ⊗ {K}_{j}^{T}}{\sqrt{{d}_{k}}} \qquad ( 5 )$$

$$\vec{a} \cdot \vec{b} = {\sum }_{i = 1}^{n} {a}_{i} {b}_{i} = {a}_{1} {b}_{1} + {a}_{2} {b}_{2} + … + {a}_{n} {b}_{n} \qquad ( 6 )$$

In formula (5), Qᵢ⊗Kⱼ^{T} represents calculating a dot product of Qᵢ and Kⱼ^{T}; the specific calculation process is as shown in formula (6), the dot product operation between two vectors is summation of products of elements in corresponding positions; dₖ represents a dimension of K; the role of \sqrt{dₖ} is scaling of the calculated similarity, making the model training process more steady; Qᵢ represents an ith query item; Kⱼ^{T} represents of a transpose of a jth key item; Attention (Qᵢ, Kⱼ) represents an attention score of the transpose Kⱼ^{T} of the ith query item Qᵢ and the jth key item; and i and j represent an ith element and a jth element in input data x. A degree of association between Q and K can be calculated by formula (5).

### (3) Calculation of Attention Weight

The similarity obtained in step (2) is normalized by the Softmax function to obtain a weight of attention (attention weight), i.e., an attention distribution.

$$Attention {Weights}_{ij} = Soft max ( Attention ( {Q}_{i} , {K}_{j} ) ) \qquad ( 7 )$$

An expression of the Softmax function is

$$\frac{{e}^{{x}_{i}}}{{\sum }_{i} {e}^{{x}_{i}}} ,$$

which is a nonlinear function, where xᵢ represents an ith element in an input vector; Σᵢe^{x}^{ᵢ} is exponential summation of all elements xᵢ; Attention Weightsᵢⱼ is a matrix of the attention distribution; and i and j represent indexes thereof in the matrix. Attention degrees of the model to different positions in the series are obtained by formula (7). Thus, the model can learn the position information in the mass spectrometric data, namely a mass-to-charge ratio, which is very important for determining a particle type.

### (4) Calculation of Weighted Sum

Weighted summation is performed on the V value with the obtained attention weight to obtain a final output.

$$Self - Attention ( X ) = {\sum }_{j = 1}^{n} Attention {Weights}_{ij} * {V}_{j} \qquad ( 8 )$$

The final output of the self-attention module is obtained by formula (8), which is series information of one dimension. Self-Attention(X) represents an output obtained by performing self-attention calculation on series X; n represents a length of the series X; and Vⱼ represents a jth element of Value obtained by linear variation.

3. MLP: The MLP is configured to receive the series data processed by Self-Attention, put the series data through an input layer, a hidden layer, and an output layer, and output a final PM classification result.

$${Y}^{′} = {σ}_{s} ( {W}^{′} \cdot {X}^{′} + {b}^{′} ) \qquad ( 9 )$$

In formula (9), σₛ represents an activation function Softmax, and an expression thereof is

$$\frac{{e}^{{x}_{i}}}{{\sum }_{i} {e}^{{x}_{i}}} ,$$

which is a nonlinear function; Y′ represents a probability of output classification; X′ represents input series data; and W′ represents a weight matrix; and b′ represents an offset vector.

4. A back propagation process in the model training process is as follows.

### (1) Forward Propagation

The data is input to the model through forward propagation, and subsequently, an initial result is obtained.

$${Z}^{l} = {W}^{( l )} {a}^{( l - 1 )} + {b}^{( l )} \qquad ( 10 )$$

$${a}^{l} = σ ( {Z}^{l} ) \qquad ( 11 )$$

$${a}_{i}^{( L )} = \frac{{e}_{i}^{{z}^{( L )}}}{{\sum }_{j = 1}^{C} {e}_{j}^{{z}^{( L )}}} \qquad ( 12 )$$

Formula (10) and formula (11) represent forward propagation from the input layer to the hidden layer; and formula (12) represents forward propagation from the hidden layer to the output layer. In formula (10), l represents a number of layers of the model; Z^{l} is an input to an lth layer; W^{(l)} represents a weight matrix of the lth layer; a^{l} represents an output of the lth layer; a^{(l-1)} represents an output of an (l-1)th layer; and b^{(l)} represents an offset vector of the lth layer. In formula (11), σ represents the Relu activation function. In formula (12), eᵢ^{z}^{^{(L)}} represents an input to an ith node; e represents a Napierian base; c represents a total number of categories;

$${\sum }_{j = 1}^{C} {e}_{j}^{{z}^{( L )}}$$

represents a sum of input exponents of all categories, j represents a serial number of a category; z^{(L)} represents a sum of inputs to an Lth layer, L represents a last layer different from other layers; and aᵢ^{(L)} represents an output of an ith category.

### (2) Calculation of Loss Function

$${L}_{loss} = - {\sum }_{i = 1}^{\begin{matrix}output \\ size\end{matrix}} {y}_{i} \cdot log {\hat{y}}_{i} \qquad ( 13 )$$

In the actual atmospheric environment, there are often more than two categories of particles, and therefore, a categorical cross-entropy (categorical_crossentropy) suitable for a multi-classification task is used as a loss function. In formula (13), Lₗₒₛₛ is the loss function; in the whole model, the loss function Lₗₒₛₛ defined in formula (13) has a fixed meaning; yᵢ represents specific classification; log [symbol] represents a probability predicted by the model; a value of outputsize depends on a classification count of the model; and i represents a series number of the classification count of the model.

### (3) Back Propagation

$${δ}^{( L )} = {a}^{( L )} - γ \qquad ( 14 )$$

$${δ}^{( l )} = {( {W}^{( l + 1 )} )}^{T} {δ}^{( l + 1 )} ⊙ {σ}^{′} ( {Z}^{( l )} ) \qquad ( 15 )$$

$$\frac{δ {L}_{loss}}{δ {W}^{( l )}} = \frac{δ {L}_{loss}}{δ {b}^{( l )}} {( {a}^{( l - 1 )} )}^{T} \qquad ( 16 )$$

Formula (14) represents back propagation from the output layer to the hidden layer; formula (15) represents back propagation between the hidden layers; and formula (16) represents calculation of a gradient, i.e., a direction in which parameters need to be updated. In formula (14), δ^{(L)} represents an error of the output layer; a^{(L)} represents an output of the model; and y represents a true result. In formula (15), δ^{(l)} represents an error item of the lth layer; W^{(l+1)} represents a weight matrix of an (l+1)th layer; [symbol] represents a Hadamard product, which represents that corresponding elements of two matrices are multiplied to obtain a new matrix; δ^{(l+1)} represents an error item of the (l+1)th layer; and σ′ represents a derivative of an activation function. In formula (16),

$$\frac{δ {L}_{loss}}{δ {W}^{( l )}}$$

represents a partial derivative of the loss function Lₗₒₛₛ to the weight matrix W^{(l)} of the lth layer, and

$$\frac{δ {L}_{loss}}{δ {b}^{( l )}}$$

represents a partial derivative of the loss function Lₗₒₛₛ to the offset vector b^{(l)} of the lth layer, and the two partial derivatives are gradients of the weight matrix and the offset matrix, respectively;

$$\frac{δ {L}_{loss}}{δ {W}^{( l )}} and \frac{δ {L}_{loss}}{δ {b}^{( l )}}$$

represent effects of the loss function Lₗₒₛₛ on the weight matrix and the offset vector at the lth layer; a^{(l)} is a result of the lth layer; and a^{(l-1)} is a result of the (l-1)th layer.

### (4) Parameter Updating

After the gradients of the weight matrix W and the offset vector b are obtained, the model parameters are updated by gradient descent.

$${W}^{( l )} = {W}^{( l )} - α \frac{δ {L}_{loss}}{δ {W}^{( l )}} \qquad ( 17 )$$

$${b}^{( l )} = {b}^{( l )} - α \frac{δ {L}_{loss}}{δ {b}^{( l )}} \qquad ( 18 )$$

Formula (17) and formula (18) represent the model parameter updating process, where a represents a learning rate.

5. Overview of training of the deep learning model

The training of the deep learning model may be explained briefly as follows: firstly, the single particle mass spectrometric data of the input PM is transferred to the model by forward propagation, and then the loss function is calculated. In the training process, the objective is to minimize the loss function. The gradient is calculated by back propagation. Subsequently, the weight matrix W and the offset vector b of the mode are updated by gradient descent. This process is iterated repeatedly until a stop condition is met (e.g., a maximum number of iterations is reached or the loss function converges).

The source apportionment of VOCs is firstly carried out in the PMF model. The PMF model is to decompose a sample matrix into two matrices: a source contribution matrix and a source profile matrix. Various pollution sources and their contribution rates and time series are determined by least-squares operation, while the value of an objective function Q is required to be as small as possible. Subsequently, the time series (contribution spectrum) of the factors is combined with the PM apportionment result, and automatic source identification is realized.

A relevant operation formula of the model is as follows:

$${x}_{ij} = {\sum }_{k = 1}^{p} {g}_{ik} {f}_{kj} + {e}_{ij} \qquad ( 19 )$$

In formula (19), i represents a number of samples; j represents a number of components; xᵢⱼ represents a volume fraction of a component j in an ith sample; gᵢₖ represents a relative contribution of a factor k to the ith sample; fₖⱼ represents a volume fraction of the component j in the factor k; eᵢⱼ represents a random error of the component j in the ith sample; the source contribution matrix is a matrix composed of gᵢₖ; the source profile matrix is a matrix composed of fₖⱼ; and p represents a total number of factors.

$$Q = {\sum }_{i = 1}^{n} {{\sum }_{j = 1}^{m} [ \frac{{x}_{ij} - {\sum }_{k = 1}^{p} {g}_{ik} {f}_{kj}}{{u}_{ij}} ]}^{2} \qquad ( 20 )$$

$$U = \sqrt{{( EF \times C )}^{2} + {( 0.5 \times MDL )}^{2}} \qquad ( 21 )$$

In formula (20), n represents a number of samples; m represents a number of components; and uᵢⱼ represents the uncertainty of a component j in an ith sample, with a calculation formula being as shown in formula (21). In formula (21), U represents the uncertainty; C represents concentration; EF represents the uncertainty, which is generally set to 5-20%; and MDL represents an instrument detection limit of the substance.

The calculation process of the PMF model is described above. The PMF model is to decompose the data of various pollutants with the time series into two matrices: contribution spectrum and profile spectrum, based on the matrix decomposition principle, where the contribution spectrum has the time series information, indicating a contribution degree of this source to a certain pollutant at this moment; and the profile spectrum contains contents of pollutants emitted by a certain pollution source. The profile spectrum can be multiplied by the contribution spectrum to approach the kinds and contents of various pollutions observed with time in reality.

The present disclosure presents the calculation process of the PMF model. After the PMF model is run, contribution spectra (g) and profile spectra (f) of a plurality of unknown sources may be obtained, where g represents a partake rate of the source, and f represents the category of the source. The category of the source needs to be identified by a person in past use. The present disclosure is based on the correlation of the time series, as shown in formula (22), to realize automatic source identification, where the deep learning model of the particle part is also developed by the present disclosure. For the VOCs part, the PMF model is adopted to obtain the VOCs factors of the pollution sources and the time series thereof, i.e., the profile and the contribution spectrum. The present disclosure has an improvement on the identification of the running result of the PMF model. Specifically, the source needs to be judged by a person after the PMF model is run in the past. The present disclosure can realize automatic determination based on the correlation calculation. In the present disclosure, the factors of the VOCs pollution sources correspond to the previous profile spectrum, and the time series corresponds to the previous contribution spectrum.

The time series of the VOCs factors is obtained by running the PMF model. Correlation analysis is performed on the PM source apportionment result and the time series of the VOCs factors. The Pearson correlation coefficient is calculated by a formula as shown in formula (22):

$$ρ = \frac{\sum ( {X}_{i} - \overline{X} ) ( {Y}_{i} - \overline{Y} )}{\sqrt{\sum {( {X}_{i} - \overline{X} )}^{2} \sum {( {Y}_{i} - \overline{Y} )}^{2}}} \qquad ( 22 )$$

In formula (22), ρ represents the Pearson correlation coefficient; Xᵢ and Yᵢ represent ith observed values in two source apportionment result time series, respectively; and X̅ and Y̅ represent mean values of the two source apportionment result time series, respectively. When the Pearson correlation coefficient is higher than a certain value, it indicates that the two groups of source apportionment results have a correlation in time series and can be attributed to the same pollution source, thereby realizing the collaborative source apportionment of the PM and the VOCs and providing technical support for the collaborative control of the pollution sources in the atmospheric environment. The present disclosure involves the collaborative use of the deep learning algorithm and the PMF algorithm. The advantage of realizing refined source apportionment of the deep learning algorithm is applied to the source apportionment of the VOCs. The apportionment result finally obtained by the method of the present disclosure is which PM and VOCs belong to the same pollution source. The source of PM and VOCs can be determined by the method of the present disclosure. According to the apportionment result, different contribution degrees of a certain pollution source to the PM and the VOCs can be assessed, and what pollution source has a prominent contribution can be identified, providing support for scientifically controlling the effects of the sources on the atmospheric environment in future. Meanwhile, the problem of subjectivity in VOCs identification is solved, and an idea of assessing the efficiency of atmosphere control (the PM pollution may decrease and the VOCs may increase for a certain source) is provided.

At present, for the source apportionment of the VOCs and the PM are given using different models, and there is no algorithm model capable of simultaneously apportioning the PM and the VOCs. In view of this, the present disclosure provides a collaborative apportionment technique for VOCs based on single particle mass spectrometry. By analyzing the correlation between the source apportionment results of the PM and the VOCs, the automatic distribution of the pollution sources is realized, the subjectivity is effectively reduced, and the time taken by the source apportionment is reduced. Moreover, benefited by the deep learning model used in the source apportionment of the PM, the defined time series of the pollution sources can be obtained, thereby assisting with the refined source apportionment of the VOCs. Furthermore, the present disclosure solves the problems that the existing source apportionment technique for VOCs takes a long time in the apportionment process and introduces the subjective factor due to artificial judging of the sources, and that the refined source apportionment technique for VOCs is not mature and cannot identify the VOCs factors of similar sources.

The main feature of this solution is the joint source apportionment work of VOCs and PM. Based on the existing technology, results of separate apportionment of VOCs sources often failed to distinguish mobile sources in detail, such as road vehicles, non-road construction machinery, and ships. According to the above solution, the source of PM can be finely analyzed by SPAMS and deep learning algorithms. Based on the concept of homologous emissions, the correlation coefficient can be used to link VOCs and PM for collaborative source apportionment. For air pollution control in urban area, the present disclosure can effectively identify the sources of PM and VOCs, thereby providing technical support for government policies and governance measures. In some embodiments, regarding a certain category of pollution source (for example, road vehicles) that is determined as the common source of VOCs and PM, when it is found that its contribution to the concentration of PM and VOCs in the atmosphere is relatively high (for example, exceeding the daily average), the VOCs collaborative source apportionment system may send a signal to the annunciator to issue a warning, reminding government departments to take a series of measures, such as increasing traffic police to alleviate road congestion, increasing the frequency of sprinkler truck operations, and encouraging the public to travel by public transportation, thereby reducing the contribution of road vehicle emissions to the concentration of pollutants in the atmosphere.

This solution can also be used to monitor the emissions of the specific industries, facilitating government departments to verify and supervise pollution emissions from enterprises. In some embodiments, the identified pollution source is an industrial source, such as textile, chemical, and plastic manufacturing, etc. The system can send a category of the common pollution source to the industrial power grid, and in response to receiving the category, the power grid adjusts the electrical power supplied to the factory corresponding to the pollution source category, so as to restrict the producing and manufacturing activities of the factories that emit pollution.

The key technical means adopted in the present disclosure are as follows: 1, the calculation of the Pearson correlation coefficient is performed on the source apportionment result time series obtained by two models to realize the collaborative apportionment of the pollution sources. 2, the deep learning model composed of the 1D-CNN, the Self-Attention, and the MLP is used, and refined source apportionment of the VOCs is carried out based on the correlation of the time series. The present disclosure can establish the unified source apportionment perspective of the PM and the VOCs to a certain extent with the correlation in time series. Specifically, by the single particle mass spectrometric source apportionment technique, the time change of the aerosol of a certain specific source can be obtained and then subjected to time correlation analysis with the factors of the VOCs obtained in the PMF model so that the common pollution source can be identified. That is to say, the present disclosure provides a method for identifying the common pollution source of the PM and the VOCs in combination with the single particle mass spectrometric source apportionment technique, the PMF model, and the time correlation analysis. The problem of separately performing the source apportionment on the PM and the VOCs because there is no method capable of identifying the common pollution source of the PM and the VOCs at present is solved. By the method for identifying the common pollution source of the PM and the VOCs in combination with the single particle mass spectrometric source apportionment technique, the PMF model, and the time correlation analysis, the present disclosure realizes the collaborative source apportionment of the PM and the VOCs and identifies the common pollution source of the PM and the VOCs, providing technical support for the collaborative control of the pollution sources in the atmospheric environment.

### EXAMPLE 2

A computer program product includes a computer program which can be executed by a processor, implements the steps of the method for collaborative source apportionment of VOCs in Example 1.

### EXAMPLE 3

A computer-readable storage medium stores a computer program which can be executed by a processor, implements steps of the method for collaborative source apportionment of VOCs in Example 1.

### EXAMPLE 4

A computer device includes a processor, a memory, an input/output (I/O) interface, and a communication interface. The processor, the memory, and the input/output interface are connected through a system bus, and the communication interface is connected to the system bus through the input/output interface. The processor of the computer device is configured to provide computing and control capabilities. The memory of the computer device includes a nonvolatile storage medium and an internal memory. The non-volatile storage medium stores an operating system, a computer program, and a database. The internal memory provides an environment for operation of the operating system and the computer program in the nonvolatile storage medium. The database of the computer device is configured to store pending transactions. The input/output interface of the computer device is configured to exchange information between the processor and an external device. The communication interface of the computer device is configured to communicate with an external terminal through a network. The computer program, when executed by a processor, can implement the steps of the method for collaborative source apportionment of VOCs in Example 1.

Particular examples are used herein for illustration of principles and implementation modes of the present disclosure. The descriptions of the above embodiments are merely used for assisting in understanding the method of the present disclosure and its core ideas. In addition, those of ordinary skill in the art can make various modifications in terms of particular implementation modes and the scope of application in accordance with the ideas of the present disclosure. In conclusion, the content of the description shall not be construed as limitations to the present disclosure.

What is claimed is:

1. A method for collaborative source apportionment of volatile organic compounds (VOCs), comprising:
monitoring, by a single particle aerosol mass spectrometry, a particulate matter (PM) in an atmospheric environment at a target area for a period of time, to obtain target single particle mass spectrometric data;
monitoring, by an on-line VOCs monitoring instrument, VOCs in the atmospheric environment in the target area for the period of time, to obtain target VOCs monitoring data;
inputting the target single particle mass spectrometric data and the target VOCs monitoring data into a pollution source identifying device to identify a category of a common pollution source of the PM and the VOCs in the target area for the period of time, wherein the source identifying device comprises a processor and a memory having an optimized single particle classification model and a positive matrix factorization (PMF) model stored therein and is configured for;
inputting the target single particle mass spectrometric data to the optimized single particle classification model for apportionment, and analyzing pollution source categories of the target single particle mass spectrometric data using the optimized single particle classification model to output a time series of the pollution source categories contributing to the PM; wherein the optimized single particle classification model is a deep learning model based on a one-dimensional convolutional neural network, a self-attention mechanism, and a multi-layer perceptron;
inputting the target VOCs monitoring data to the positive matrix factorization (PMF) model for apportionment, and outputting VOCs factors of the pollution source categories and a time series of the VOCs factors by the PMF model;
performing correlation calculation on the time series of the pollution source categories contributing to the PM and the time series of the VOCs factors to obtain a correlation coefficient; and
attributing the PM and the VOCs factor having a correlation coefficient higher than a predetermined threshold to a same pollution source category, thereby identifying the category of the common pollution source of the PM and the VOCs in the target area for the period of time.

2. The method for collaborative source apportionment of VOCs according to claim 1, wherein the optimized single particle classification model is obtained by following step:
obtaining a local pollution library, wherein the local pollution library comprises single particle mass spectrometric data of known pollution sources;
classifying known pollution sources into a plurality of pollution source categories based on industrial types;
establishing a single particle classification model, wherein the single particle classification model is a deep learning model based on a one-dimensional convolutional neural network, a self-attention mechanism, and a multi-layer perceptron;
training and optimizing the single particle classification model with single particle mass spectrometry data labeled with the plurality of pollution source categories to obtain the optimized single particle classification model.

3. The method for collaborative source apportionment of VOCs according to claim 1, wherein the method further comprises following steps:
adjusting an electrical power supplied by an industrial power grid to a factory corresponding to the category of the common pollution source to limit generation of the PM and the VOCs by the factory.

4. The method for collaborative source apportionment of VOCs according to claim 1, wherein the one-dimensional convolutional neural network, the self-attention mechanism, and the multi-layer perceptron are connected in sequence;
the one-dimensional convolutional neural network is configured to extract a local feature from input single particle mass spectrometric data;
the self-attention mechanism is configured to calculate a plurality of features extracted by the one-dimensional convolutional neural network; and
the multi-layer perceptron is configured to receive series data processed by the self-attention mechanism, put the series data through an input layer, a hidden layer, and an output layer, and output a final PM classification result.

5. The method for collaborative source apportionment of VOCs according to claim 4, wherein calculating, by the self-attention mechanism, a plurality of features extracted by the one-dimensional convolutional neural network specifically comprises:
for the plurality of features extracted by the one-dimensional convolutional neural network, calculating Query, Key, and Value values by linear variation;
calculating a similarity between the Query and Key values based on a dot product formula;
normalizing the similarity between the Query and Key values by a Softmax function to obtain a weight of attention; and
performing weighted summation on the Value values with the weight of the attention to obtain a final output, wherein the final output is one-dimensional series information.

6. The method for collaborative source apportionment of VOCs according to claim 1, wherein when training and optimizing the single particle classification model with the local pollution library, a categorical cross-entropy suitable for a multi-classification task is used as a loss function.

7. The method for collaborative source apportionment of VOCs according to claim 1, wherein the performing correlation calculation on the time series of the pollution sources contributing to the PM and the time series of the VOCs factors to obtain a correlation coefficient specifically comprises:
performing correlation calculation on the time series of the pollution sources contributing to the particle matter and the time series of the VOCs factors by a Pearson correlation coefficient calculation formula to obtain a Pearson correlation coefficient.

8. A computer apparatus, comprising a memory, a processor, and a computer program stored on the memory and runnable on the processor, wherein the processor is configured to execute the computer program to implement steps of the method for collaborative source apportionment of VOCs according to claim 1.

9. The computer apparatus according to claim 8, wherein the one-dimensional convolutional neural network, the self-attention mechanism, and the multi-layer perceptron are connected in sequence;
the one-dimensional convolutional neural network is configured to extract a local feature from input single particle mass spectrometric data;
the self-attention mechanism is configured to calculate a plurality of features extracted by the one-dimensional convolutional neural network; and
the multi-layer perceptron is configured to receive series data processed by the self-attention mechanism, put the series data through an input layer, a hidden layer, and an output layer, and output a final PM classification result.

10. The computer apparatus according to claim 9, wherein calculating, by the self-attention mechanism, a plurality of features extracted by the one-dimensional convolutional neural network specifically comprises:
for the plurality of features extracted by the one-dimensional convolutional neural network, calculating Query, Key, and Value values by linear variation;
calculating a similarity between the Query and Key values based on a dot product formula;
normalizing the similarity between the Query and Key values by a Softmax function to obtain a weight of attention; and
performing weighted summation on the Value values with the weight of the attention to obtain a final output, wherein the final output is one-dimensional series information.

11. The computer apparatus according to claim 8, wherein when training and optimizing the single particle classification model with the local pollution library, a categorical cross-entropy suitable for a multi-classification task is used as a loss function.

12. The computer apparatus according to claim 8, wherein the performing correlation calculation on the time series of the pollution sources contributing to the PM and the time series of the VOCs factors to obtain a correlation coefficient specifically comprises:
performing correlation calculation on the time series of the pollution sources contributing to the particle matter and the time series of the VOCs factors by a Pearson correlation coefficient calculation formula to obtain a Pearson correlation coefficient.
